(19) United States
(12) Patent Application Publication
(10) Pub. No.: US 2026/0000095 A1
(43) Pub. Date: Jan. 1, 2026

(54) RUMINANT FEED

(71) Applicant: Unibio Tech Science A/S, Odense SØ (DK)

(72) Inventor: Ebbe Busch LARSEN, Odense SØ (DK)

(21) Appl. No.: 18/992,043

(22) PCT Filed: Jul. 4, 2023
(86) PCT No.: PCT/EP2023/068425

§ 371 Date: Jan. 7, 2025

(30) Foreign Application Priority Data
Jul. 8, 2022 (DK) PA202200666
May 22, 2023 (DK) PA202330039
Jun. 19, 2023 (DK) PA202330088

(51) Int. Cl. A23K 10/18; A23K 20/147; A23K 50/15; C12N 1/20

(52) U.S. Cl. CPC A23K 10/18; A23K 20/147; A23K 50/15; C12N 1/20

(57) ABSTRACT

The present invention relates to an ingredient, wherein the ingredient comprises a viable methanotroph in combination with at least one oxygen agent, and a feed product comprising the ingredient.

### TECHNICAL FIELD OF THE INVENTION

The present invention relates to an ingredient for a ruminant feed product. In particular, the present invention relates to an ingredient for a ruminant feed product capable of reducing the methane emission of the ruminant consuming the feed product resulting in environmental benefits.

### BACKGROUND OF THE INVENTION

In ruminants, methane is produced mostly by enteric fermentation where microbes decompose and ferment plant materials, such as celluloses, fibre, starches, and sugars, in their digestive tract or rumen. Enteric methane is one by-product of this digestive process and is expelled by the animal through burping.

Methane (CH₄) is recognized as the second most important greenhouse gas emitted from anthropogenic sources. Ruminants contribute to approximately one-quarter of all anthropogenic sources of CH₄ emissions. In 2005, the Kyoto protocol came into effect and the signatories committed to reduce greenhouse gas emissions to an agreed level.

As a result of the Kyoto protocol, the focus in animal science is to reduce the overall production of CH₄ production from ruminants, and attention has shifted toward its contribution to climate change and global warming.

In addition to emissions of greenhouse gas, CH₄ produced by ruminants represents an energy loss to the animal. Depending on the level of feed intake, diet composition, and other factors, CH₄ energy loss may vary from 2 to 12% of gross energy intake.

The cow's digestive tract consists of the following:
Mouth
Esophagus
A four-compartment stomach, which includes
The rumen (paunch)
The reticulum (“honeycomb”)
The omasum (“manyplies”)
The abomasum (“true stomach”)
Small intestine
Large intestine

### The Rumen

The rumen (on the left side of the animal) is the largest stomach compartment and consists of several sacs. It can hold 25 gallons or more of material depending on the size of the cow. Because of its size, the rumen acts as a storage or holding vat for feed.

Aside from storage, the rumen is also a fermentation vat. The rumen's environment favours the growth of microbes. These microbes digest or ferment feed within the rumen and make volatile fatty acids (VFAs). The volatile fatty acids may be the cow's main energy source. The rumen absorbs most of the VFAs from fermentation.

A good blood supply to the rumen walls improves absorption of VFAs and other digestion products. Tiny projections (papillae) line the rumen, which increases the rumen's surface area and the amount it can absorb.

The reticulum is a pouch-like structure in the forward area of the body, close to the heart. The tissues in the reticulum form a network similar to a honeycomb. A small tissue fold lies between the reticulum and rumen, but the two aren't separate compartments. Together they're called the rumino-reticulum.

Heavy or dense feed and metal objects eaten by the cow drop into this compartment. Nails and other sharp objects may work into the tissue and cause “hardware disease.” You can use magnets to prevent disease or correct the problem through surgery. Leaving it untreated may lead to infection and possibly death.

The omasum is a globe-shaped structure containing leaves of tissue (like pages in a book). It absorbs water and other substances from digestive contents. Feed material (ingesta) between the leaves will be drier than ingesta found in the other compartments.

The abomasum is the only compartment lined with glands. These glands release hydrochloric acid and digestive enzymes, needed to breakdown feeds.

Digestion of feed by ruminants may produce significant amount of gases mainly CO₂ and methane and the amount of gas produced may be determined as volumetric flow rate, as cubic meter per hour (m³/h). On average a ruminant, in particular a cow, produces 30 to 50 cubic meter per hour (m³/h)—30 to 50 quarts of gas per hour in the rumen—of gas in the rumen.

Hence, an ingredient or a feed product that would reduce ruminant emission of one or more greenhouse gasses, in particular methane, when digested would be advantageous, and in particular an ingredient or a feed product that that, when digested, makes ruminants more productive, more environmentally friendly and/or provide nutritional value would be advantageous.

### SUMMARY OF THE INVENTION

Thus, an object of the present invention relates to an ingredient for a ruminant feed product.

In particular, it is an object of the present invention to provide an ingredient and a feed product that solves the environmental problems with ruminant emission of greenhouse gasses, in particular methane and carbon dioxide (CO₂), in particular emission of methane.

Thus, one aspect of the invention relates to an ingredient, in particular an ingredient for a feed product, preferably a ruminant feed product, wherein the ingredient comprises a viable methanotroph.

Another aspect of the present invention relates to an ingredient, wherein the ingredient comprises a viable methanotroph in combination with at least one oxygen agent.

Yet another aspect of the present invention relates to a feed product comprising a viable methanotroph.

A further aspect of the present invention relates to a feed product (in particular a ruminant feed product), comprising at least one oxygen agent.

A yet an aspect of the present invention relates to a feed supplement, in particular a ruminant feed supplement, comprising at least one oxygen agent.

Still another aspect of the present invention relates to a feed product comprising the ingredient according to the present invention.

A further aspect of the present invention relates to the use of the ingredient according to the present invention and/or use of the feed product according to the present invention for increasing the milk production in a ruminant.

Yet a further aspect of the present invention relates to a method and/or a system for increasing the oxygen concentration in the rumen of ruminant by administering an ingredient or a feed product comprising a oxygen agent to a ruminant.

The present invention will now be described in more detail in the following.

### DETAILED DESCRIPTION OF THE INVENTION

Accordingly, both dietary and animal factors may play an important role in CH₄ production and feed/forage composition, and proportion may be an important factor influencing CH₄ production in ruminants.

The inventor of the present invention surprisingly found that a specific composition of ingredients in the feed product may provide a reducing effect on emission of methane from the ruminant for the environmental benefit and at the same time an improved nutritional effect and lactation may be observed.

Thus, in a preferred embodiment of the present invention relates to an ingredient for a feed product, preferably a ruminant feed product, wherein the ingredient comprises a viable methanotroph.

Preferably, the ingredient may further comprise at least one oxygen agent.

A preferred embodiment of the present invention relates to an ingredient, wherein the ingredient comprises a viable methanotroph in combination with at least one oxygen agent.

The oxygen agent may be selected from a readily available oxygen compound, an oxygen-precursor compound, an oxygen-developing agent, or a combination hereof.

The inventor of the present invention found that addition of viable methanotroph may be suitable for reducing the emission of methane from the ruminant into the atmosphere—and thus constitute a significant represent a significantly lower impact on the environmental than in the past.

This reduced emission of methane and reduced impact on the environment may be even greater, significantly greater, if oxygen is made available to the methanotroph as the conversion of methane was significantly improved.

In the rumen of ruminants is anaerobic and oxygen may be present only in low concentrations in both the rumen liquid and rumen gas phases.

The inventor of the present invention found that by increasing the oxygen concentration in the rumen of ruminant, and in particular in the rumen liquid, the reduction in methane emission from the ruminant may be significantly reduced.

Thus, a preferred embodiment of the present invention relates to a method and/or a system for increasing the oxygen concentration in the rumen of ruminant by administering an ingredient or a feed product comprising an oxygen agent to a ruminant.

In an embodiment of the present invention the oxygen agent according to the present invention may increase the oxygen available to the growth and/or activity of the viable methanotroph when in the rumen of the ruminant. Preferably, the oxygen agent according to the present invention may increase the oxygen in the rumen liquid to be available to the growth and/or activity of the viable methanotroph when in the rumen of the ruminant.

The oxygen agent according to the present invention may include any means for providing or supplying oxygen to the gastrointestinal tract, in particular the rumen, of a ruminant.

The oxygen may be supplied in many different forms, however in the rumen of the ruminant the oxygen may preferably be available as dioxygen (O₂).

Preferably, oxygen may be a readily available oxygen compound, an oxygen-precursor compound, an oxygen-developing agent, or a combination hereof.

A readily available oxygen may be dioxygen (O₂) that may be included into the ingredient of the present invention or into the feed product according to the present invention and made available to the viable methanotrophs when provided to the gastrointestinal tract, in particular in the rumen, of the ruminant.

Dioxygen (O₂) may be supplied to the gastrointestinal tract, in particular in the rumen, of the ruminant by encapsulation of the dioxygen (O₂) allowing release of the dioxygen in the gastrointestinal tract, in particular in the rumen.

Another way to supply dioxygen to the gastrointestinal tract, in particular to the rumen, of the ruminant may be by using a carrier material holding and transporting the dioxygen, until released in the gastrointestinal tract, in particular in the rumen. Carrier materials may be a solid carrier like an aerogel, a zeolite, a carbonaceous material (e.g. pyrolytic carbon), calcium carbonate, chalk, or other catalysts providing chemisorbed oxygen.

Additionally, the readily available oxygen may be dioxygen (O₂) and the dioxygen (O₂) may be included into the ingredient of the present invention or into the feed product according to the present invention by mixing the dioxygen (O₂).

In an embodiment of the present invention the dioxygen may be mixed with the methanotroph, e.g. the dioxygen may be mixed with the methanotroph in a container. Preferably, the container may be provided with means for continuous mixing of the dioxygen and the methanotroph. This continuous mixing may ensure improved solubility of the oxygen and improved availability og the dioxygen for the methanotrophs.

The mixture of dioxygen and methanotrophs may be mixed with a basic feed material, either mixed as individual components or sequentially, e.g. by first mixing dioxygen with the methanotroph followed by mixing with the basic feed material.

When mixing the ingredients as mentioned above agitation may be preferred to reduce loss of oxygen from the mixture and therefore increase the presence (and/or transfer) of dioxygen feed product to increase the content of dioxygen in gastrointestinal tract, in particular to the rumen, of a ruminant.

Preferably, agitation of the feed product comprising the ingredient according to the present invention and the basic feed material may be stopped at most 2 hours before the feed product may be fed to the ruminant, such as at most 1.5 hours, e.g. at most 1 hour, such as at most 50 minutes, e.g. at most 40 minutes, such as at most 30 minutes, e.g. at most 20 minutes, such as at most 10 minutes, e.g. at most 5 minutes, such as at most 3 minutes, e.g. at most 1 minutes.

In the present invention the feed product may be fed to the ruminant when made available to the ruminant, not necessary when taken up by the ruminant.

Agitation of the feed product comprising the ingredient according to the present invention and the basic feed material may be stopped at most 2 hours before the feed starts to be consumed by the ruminant, such as at most 1.5 hours, e.g. at most 1 hour, such as at most 50 minutes, e.g. at most 40 minutes, such as at most 30 minutes, e.g. at most 20 minutes, such as at most 10 minutes, e.g. at most 5 minutes, such as at most 3 minutes, e.g. at most 1 minutes.

The oxygen-precursor compound may be a compound the may be transported to the gastrointestinal tract, in particular to the rumen, of a ruminant as one or more oxygen atom, preferably as part of a molecular compound, and when arrived at the gastrointestinal tract, in particular to the rumen, of a ruminant, the oxygen-precursor compound may be converted and/or reacted into a dioxygen (O₂) and become available to the methanotroph in the gastrointestinal tract, in particular to the rumen, of a ruminant.

The conversion and/or reaction of the oxygen-precursor compound may be a chemical conversion/reaction, a microbial conversion/reaction, or an enzymatic conversion/reaction of the oxygen-precursor compound in the gastrointestinal tract, in particular in the rumen.

In an embodiment of the present invention the ingredient according to the present invention may consists essentially of viable methanotrophs in combination with an oxygen-precursor compound.

In a further embodiment of the present invention the oxygen-precursor compound may be hydrogen peroxide (H₂O₂) or a hydrogen peroxide generating compound. Preferably, the hydrogen peroxide generating compound may be percarbonate, like sodium percarbonate (2Na₂CO₃·3H₂O₂).

Oxygen-precursor compounds like sodium percarbonate, or sodium carbonate peroxide, may be an adduct of sodium carbonate and hydrogen peroxide and may be a colourless, crystalline, hygroscopic and water-soluble solid.

In an embodiment of the present invention the oxygen-precursor compound, e.g. sodium percarbonate, may comprise 15-60% by weight of hydrogen peroxide, such as in the range of 20-50% (w/w), e.g. 25-40% (w/w), such as in the range of 30-35% (w/w), e.g. about 32.5% by weight of the oxygen-precursor compound, e.g. sodium percarbonate, may be hydrogen peroxide.

The oxygen-precursor compounds according to the present invention, like sodium percarbonate, may dissolved in water yielding oxygen/dioxygen (O₂), i.e. like sodium percarbonate may yield a mixture of hydrogen peroxide (which eventually decomposes to water and dioxygen (O₂)), sodium cations and carbonate.

The oxygen or the dioxygen (O₂) produced from the oxygen-precursor compounds, may then according to the present invention, be available to the viable methanotroph and methane developed in the rumen of the ruminant may be degraded resulting in a reduction of the emission of methane from the ruminant.

An advantage of using hydrogen peroxide generating compounds like percarbonates (e.g. sodium percarbonate) may be that sodium percarbonate may be provided in solid form which may be easier to handle and/or keep stable during production and/or during storage.

An oxygen-developing agent may be an agent capable of converting one or more constituents in the feed product and/or in the gastrointestinal tract, in particular in the rumen, of the ruminant, into a dioxygen (O₂) compound.

In an embodiment of the present invention the oxygen-developing agent may be a microorganism or an enzyme.

In the event the oxygen-developing agent may be an oxygen-developing microorganism, the oxygen-developing microorganism may be provided together with the methanotroph in the ingredient according to the present invention, or the oxygen-developing microorganism may be provided as a separate ingredient in the feed product, either in a separate feed product or in the feed product according to the present invention.

In an embodiment of the present invention the ingredient and/or the feed product according to the present invention may comprise the combination of the oxygen-precursor compound (e.g. in the form of a oxygen containing chemical compound) and the oxygen-developing agent like a microorganism and/or an enzyme capable of converting the oxygen-precursor compound into dioxygen (O₂).

In a further embodiment of the present invention the viable microorganisms present in the ingredient according to the present invention and/or in the feed product according to the present invention may consists essentially of viable methanotrophs in combination with an oxygen-developing microorganism.

As mentioned above, it was found that by increasing the concentration of oxygen, preferably dioxygen (O₂), in the gastrointestinal tract, in particular in the rumen, of a ruminant the activity of the methanotroph may be significantly increased and the emission of methane from the ruminant to the environment may be significantly reduced.

In an embodiment of the present invention the concentration of oxygen, preferably the concentration of dioxygen (O₂), in the gastrointestinal tract, in particular in the rumen (preferably in the rumen liquid), of a ruminant may be increased to an oxygen concentration, preferably dioxygen (O₂) concentration, of at least 1 μmol/l, such as at least 2 μmol/l, e.g. at least 3 μmol/l, such as at least 4 μmol/l, e.g. at least 5 μmol/l, such as at least 7.5 μmol/l, e.g. at least 10 μmol/l, such as at least 12.5 μmol/l, e.g. at least 15 μmol/l, such as at least 25 μmol/l, e.g. at least 50 μmol/l, such as at least 75 μmol/l, e.g. at least 100 μmol/l.

In a further embodiment of the present invention the concentration of oxygen, preferably the concentration of dioxygen (O₂), in the gastrointestinal tract, in particular in the rumen (preferably in the rumen gas), of a ruminant may be increased to an oxygen concentration, preferably dioxygen (O₂) concentration, of at least 1 kPa, such as at least 2 kPa, e.g. at least 3 kPa, such as at least 4 kPa, e.g. at least 5 kPa, such as at least 7.5 kPa, e.g. at least 10 kPa.

Determination of the amount of the at least one oxygen agent to be added to the ingredient according to the present invention and/or the amount of the at least one oxygen agent to be added to the feed product according to the present invention may be determined by introducing an oxygen electrode into the gastrointestinal tract, in particular into the rumen, of the ruminant. The determination of oxygen in the rumen of a ruminant is known to the skilled person.

In an embodiment of the present invention the oxygen agent and/or the methanotroph, may be encapsulated resulting in an encapsulated oxygen agent and/or an encapsulated methanotroph or a combined encapsulated oxygen agent and methanotroph.

The encapsulation may improve control of the delivery into the digestive tract or rumen of the ruminant and/or increase stability of the oxygen agent and/or the methanotroph.

Furthermore, encapsulation may be provided to prevent damage to or degradation of probiotic bacteria during storage, exposure to water or moisture, exposure to elevated temperatures, and exposure to acidic and high bile salt environments, for example, when they pass through the gastrointestinal tract, and to increase stability of the oxygen agent and increase stability and/or viability of the methanotrophs.

The encapsulation process may provide the encapsulated material (the oxygen agent and/or the methanotroph) with a barrier to the surrounding environment. The barrier may comprise one or more polymers, preferably one or more biopolymers.

In the digestive tract or rumen of the ruminant, the barrier may subsequently be degraded to release the oxygen agent and/or the methanotroph at a target site, e.g. the rumen of the ruminant.

The degradation of the barrier may define the time of release of the encapsulated product (e.g. the oxygen agent and/or the methanotroph).

In an embodiment of the present invention the release time and/or the degradation of the barrier (resulting in release of the oxygen agent and/or the methanotroph) may be controlled by using different barrier materials, by changing the barrier thickness, by using different types of barrier materials.

The feed product according to the present invention may preferably be provided with a distribution of encapsulated oxygen agent and/or an encapsulated methanotroph or a combined encapsulated oxygen agent and methanotroph, with different release times of oxygen agent and/or methanotrophs.

Preferably, the distribution of encapsulated oxygen agent and/or encapsulated methanotroph or combined encapsulated oxygen agent and methanotroph, with different release times, may be set in order to provide full, or substantially full, release of oxygen agent and/or methanotrophs, in the period between two feedings of the ruminant. This means that if a ruminant is fed approximately every 6 hours, the release of the oxygen agent and/or methanotrophs from the distribution of encapsulated oxygen agent and/or an encapsulated methanotroph or a combined encapsulated oxygen agent and methanotroph, may be configured to release the oxygen agent and/or methanotrophs over the period of 6 hours.

In an embodiment of the present invention the barrier may be configured to provide a controlled release of the oxygen agent and/or methanotrophs from the encapsulated oxygen agent and/or the encapsulated methanotroph or the combined encapsulated oxygen agent and methanotroph to the gastrointestinal tract, in particular the rumen, of the ruminant.

Preferably, the oxygen agent may be an encapsulated oxygen agent.

The controlled release of the oxygen agent and/or methanotrophs from the encapsulated oxygen agent and/or the encapsulated methanotroph or the combined encapsulated oxygen agent and methanotroph to the gastrointestinal tract, in particular the rumen, of the ruminant, may preferably be over a period of 1-12 hours, such as over a period of 1.5-8 hours, e.g. over a period of 2-6 hours, such as over a period of 2.5-4 hours, e.g. over a period of 2.75-3 hours.

In an embodiment of the present invention the encapsulated oxygen agent and/or the encapsulated methanotroph or the combined encapsulated oxygen agent and methanotroph may be configured to provide an increased release as the ruminant may be eating and may be slightly decreasing towards the next feeding, e.g. every 12 hours.

The barrier material or the biopolymers may comprise a hydrophilic polymer material or a hydrophobic material.

The barrier material or the biopolymers may be selected from the group alginate, gelatine, starch, xanthan gum, guar gum, locust bean gum, chitosan, carrageenan gum, fat, cellulose, cellulose ester, and a combination hereof.

The encapsulated oxygen agent and/or the encapsulated methanotroph or the combined encapsulated oxygen agent and methanotroph.

The encapsulated oxygen agent may comprise two or more layers of barrier material and different thicknesses of barrier material to regulate the and/or control the release of the oxygen agent and/or the methanotroph or the combined oxygen agent and methanotroph.

In an embodiment the encapsulation according to the present invention may include encapsulation or coating of the oxygen agent and/or the methanotroph or the combined oxygen agent and methanotroph, resulting in the encapsulated oxygen agent and/or the encapsulated methanotroph or the combined encapsulated oxygen agent and methanotroph.

In an embodiment of the present invention the oxygen agent and/or the methanotroph or the combined oxygen agent and methanotroph may be provided in a capsule, a pill, a tablet, a coated tablet, a powder, a coated powder, or the like.

The capsule according to the present invention may comprise the oxygen agent in liquid form, gas form or solid form. Preferably, the capsule according to the present invention may comprise the oxygen agent in solid form.

A preferred embodiment of the present invention relates to an encapsulated oxygen agent comprising in the range of 10-10,000 mg oxygen agent per capsule (pill, or tablet), such as in the range of 25-7,500 mg oxygen agent, e.g. in the range of 50-5,000 mg oxygen agent, such as in the range of 75-2,500 mg oxygen agent, e.g. in the range of 100-2,000 mg oxygen agent, such as in the range of 150-1,500 mg oxygen agent, e.g. in the range of 200-1,000 mg oxygen agent, such as in the range of 300-750 mg oxygen agent, e.g. in the range of 350-650 mg oxygen agent, such as in the range of 400-600 mg oxygen agent, e.g. in the range of 450-550 mg oxygen agent.

Preferably, the encapsulated oxygen agent may be provided as a capsule, a pill, a tablet, or a coated powder. Preferably, the encapsulated oxygen agent may be provided as a capsule.

The capsule, the pill, the tablet, or the coated powder according to the present invention may be provided with a hydrogen peroxide compound, in particular a percarbonate compound, like sodium percarbonate.

The capsule, the pill, the tablet, the coated tablet, or the coated powder according to the present invention may be further provided with the methanotroph.

The processes of encapsulation and/or coating of the capsule, the pill, the tablet, the coated tablet, the powder, the coated powder, or the like, are described in the prior art and known to the skilled person.

Preferably, the present invention may relate to a feed supplement, in particular a ruminant feed supplement, comprising at least one oxygen agent.

In an embodiment of the present invention the feed supplement may be provided with an oxygen agent. Preferably, the feed supplement may be made available to the animal, in particular made available to a ruminant, separate from the feed provided to the animal, in particular to a ruminant.

The feed supplement may be provided as a solid block comprising the oxygen agent, preferably the oxygen agent as described herein.

The solid block may be provided with a flavouring agent attracting the animal, in particular the ruminant, to lick the solid block, releasing the oxygen agent to the mouth of the animal and allowing the oxygen agent to be transported to the rumen of the animal where the oxygen agent may be released.

The methanotroph may be a methanotrophic microorganism.

In an embodiment of the present invention the methanotrophic microorganism may be a methanotrophic bacteria, a methanotrophic yeast; a methanotrophic fungus; or an a methanotrophic alga. Preferably, the methanotrophic microorganism may be a methanotrophic bacteria.

The methanotrophic bacteria may be selected from the group consisting of Methylomonas, Methylobacter, Methylococcus, Methylosinus, and mixtures thereof.

Preferably, the methanotrophic bacteria may be Methylococcus capsulatus.

The methanotrophic bacteria may be added as a) an inoculum comprising essentially methanotrophic bacteria and/or b) an isolated methanotrophic bacteria or spore.

In a preferred embodiment of the present invention the methanotrophic microorganism may be a viable methanotrophs, such as a viable methanotrophic microorganism, in particular a viable methanotrophic bacteria.

The viable methanotrophs may be a microorganism which may be able to have an active metabolism.

In an embodiment of the present invention the ingredient, and/or the feed product, may comprise at least 25% viable methanotrophs, relative to the total number of methanotrophs in the ingredient and/or in the feed product, such as at least 30% viable methanotrophs, e.g. at least 40% viable methanotrophs, such as at least 50% viable methanotrophs, e.g. at least 60% viable methanotrophs, such as at least 70% viable methanotrophs, e.g. at least 80% viable methanotrophs, such as at least 90% viable methanotrophs, e.g. at least 95% viable methanotrophs, such as at least 97% viable methanotrophs, e.g. at least 98% viable methanotrophs.

In one embodiment of the invention the ingredient, and/or the feed product, may comprises viable microorganisms, in particular viable methanotrophs, in a concentration from 10³ to 10¹³ colony forming units of microorganisms per gram, such as in a concentration of 10⁴ to 10¹⁰ colony forming units of microorganisms per gram, e.g. in a concentration of 10⁵ to 10⁹ colony forming units of microorganisms per gram.

In one embodiment of the invention the ingredient and/or the feed product may comprise viable methanotrophs in a concentration of at least 10³ colony forming units of microorganisms per gram. More specifically an ingredient and/or a feed product comprising of at least 10⁴ colony forming units of microorganisms per gram, more specifically an ingredient and/or a feed product comprising at least 10⁵ colony forming units of microorganisms per gram, more specifically an ingredient and/or a feed product comprising at least 10⁶ colony forming units of microorganisms per gram, more specifically an ingredient and/or a feed product comprising at least 10⁷ colony forming units of microorganisms per gram.

Preferably, the microorganisms present in the ingredient of the present invention consist essentially of methanotrophs.

In an embodiment of the present invention the viable microorganisms present in the ingredient consists essentially of viable methanotrophs.

In the context of the present invention, the term “consists essentially of”, relates to a limitation of the scope of a feature or a claim to the specified features or steps, and those features or steps, not mentioned and which do not materially affect the basic and novel characteristic(s) of the claimed invention.

The microorganisms present in the ingredient of the present invention may consist of methanotrophs.

In a further embodiment of the present invention the viable microorganisms present in the ingredient consists of viable methanotrophs.

In the context of the present invention the term “consists of” may relate to an ingredient or a feed product comprising no further microorganism in the ingredient or in the feed product, apart from the ones following the wording.

In an embodiment of the present invention the viable methanotroph constitute at least 40% of the total number of viable microorganisms in the ingredient (based on cell count), such as at least 50%, e.g. at least 60%, such as at least 70%, e.g. at least 80%, such as at least 90%, e.g. at least 95%, such as at least 98%, e.g. at least 99%.

In a further embodiment of the present invention the ingredient further comprises a single cell protein (SCP) or a derivative hereof.

The single cell protein (SCP) or a derivative hereof may preferably comprise no viable microorganism. Thus, the single cell protein (SCP) or a derivative hereof may not be able to perform an active metabolism.

Preferably, the ingredient comprises at least 1% (w/w) single cell protein (SCP), such as at least 2.5% (w/w), e.g. at least 5% (w/w), such as at least 7.5% (w/w), e.g. at least 10% (w/w), such as at least 15% (w/w), e.g. at least 20% (w/w), such as at least 25% (w/w), e.g. at least 30% (w/w), such as in the range of 1-30% (w/w), e.g. in the range of 5-20% (w/w), such as in the range of 10-20% (w/w), e.g. in the range of 12-16% (w/w).

Single-cell proteins (SCP) may be an edible unicellular microorganism. The biomass or protein extract from pure or mixed cultures of algae, yeasts, fungi or bacteria may be used as single cell protein and may be used as an ingredient or a substitute for protein-rich foods and may be suitable for human consumption or as animal feeds, such as a ruminant feed.

In order to further improve the effect of the ingredient and the feed product according to the present invention, the pH of the ingredient and/or the feed product may be adjusted to benefit and/or favour the activity of the growth and activity of the methanotroph when provided to the digestive system of the ruminant.

In an embodiment of the present invention the ingredient may comprise a pH in the range of pH 5.5-9.0, such as in the range of pH 6.0-8.5, e.g. in the range of pH 6.5-8.0, such as in the range of pH 6.8-7.5.

Preferably, the ingredient and/or the feed product according to the present invention may be capable of providing a pH in the digestive system of the ruminant in the range of pH 5.5-9.0, such as in the range of pH 6.0-8.5, e.g. in the range of pH 6.5-8.0, e.g. in the range of pH 6.8-7.5.

The pH, or the pH adjustment, may be provided by addition of an alkaline, an acid or a buffer. Preferably the alkaline, the acid or the buffer may be an organic alkaline, an organic acid or an organic buffer.

Preferably, the salt form of the buffer, preferably the salt form of the organic buffer, may be based on sodium calcium or potassium.

In an embodiment of the present invention the ingredient may be a liquid ingredient, an aqueous ingredient or a dry ingredient.

The aqueous ingredient may have a solid content in the range of 2-50% solids, such as in the range of 3-25% solids, e.g. in the range of 4-12% solids, such as in the range 5-10% solids.

The dry ingredient may have a moisture content in the range of 2-50% moisture, such as in the range of 3-25% moisture, e.g. in the range of 4-12% moisture, such as in the range 5-10% moisture.

A preferred embodiment of the present invention relates to a feed product comprising a viable methanotroph.

Preferably, the viable microorganisms present in the feed product according to the present invention may consists essentially of viable methanotrophs.

Even more preferably, the viable microorganisms present in the feed product according to the present invention consists of viable methanotrophs.

A preferred embodiment of the present invention relates to a feed product comprising the ingredient according to the present invention.

Preferably, the feed product is a ruminant feed product.

In an embodiment of the present invention the feed product may be formulated into a ruminant feed product.

Preferably, the feed product may comprise 1-15% (w/w) of the ingredient according to the present invention, e.g. 2-12% (w/w); such as 3-10% (w/w); e.g. 4-8% (w/w), such as about 5% (w/w).

In an embodiment of the present invention the feed product may comprise 1-15% (w/w) methanotroph (preferably, viable methanotroph), e.g. 2-12% (w/w) methanotroph (preferably, viable methanotroph); such as 3-10% (w/w) methanotroph (preferably, viable methanotroph); e.g. 4-8% (w/w) methanotroph (preferably, viable methanotroph), such as about 5% (w/w) methanotroph (preferably, viable methanotroph).

Preferably, the methanotroph present in the ingredient and/or in the feed product may be a viable methanotroph.

In an embodiment of the present invention the ingredient or the feed product according to the present invention may comprise a combination of viable methanotroph and non-viable methanotrophs. When the ingredient or the feed product according to the present invention may comprise a combination of viable methanotroph and non-viable methanotrophs, the feed product comprises 1-15% (w/w) methanotroph (as described above), e.g. 2-12% (w/w) methanotroph; such as 3-10% (w/w) methanotroph; e.g. 4-8% (w/w) methanotroph, such as about 5% (w/w) methanotroph.

Accordingly, in one embodiment of the invention, the proportion of the ingredient in the feed product may be within the range of 0.1-99.9 vol-%, 1-99 vol-%, 5-95 vol-%, 10-90 vol-%, 15-85 vol-%, 20-80 vol-%, 25-75 vol-%, 30-70 vol-%, 35-65 vol-%, 40-60 vol-%, 45-55 vol-%, preferably around 1-5 vol-%, such as 2-4 vol-%. Thus, the feed product may be provided with a concentration of methanotrophic bacteria sufficient to reduce amount of methane emanating from the digestive tract of ruminants (or livestock).

A preferred embodiment of the present invention relates to a feed product (in particular a ruminant feed product) comprising an oxygen agent according to the present invention.

In an embodiment of the present invention the feed product is provided with 0.0001-0.3 kg oxygen agent, in particular an oxygen-precursor compound, preferably, a hydrogen peroxide (H₂O₂) or a hydrogen peroxide generating compound (preferably a percarbonate like sodium percarbonate) per kg feed, such as 0.0005-0.2 kg per kg feed, e.g. 0.001-0.1 kg per kg feed, such as 0.005-0.075 kg per kg feed, e.g. 0.01-0.05 kg per kg feed, such as 0.0125-0.03 kg per kg feed, e.g. 0.015-0.02 kg per kg feed.

In a further embodiment of the present invention the feed product is provided with 0.1-25% (w/w) oxygen agent, in particular an oxygen-precursor compound, preferably, a hydrogen peroxide (H₂O₂) or a hydrogen peroxide generating compound (preferably a percarbonate like sodium percarbonate) relative to the weight of the feed product, such as in the range of 0.2-20% (w/w), e.g. in the range of 0.3-15% (w/w), such as in the range of 0.4-10% (w/w), e.g. in the range of 0.5-8% (w/w), such as in the range of 0.6-6% (w/w), e.g. in the range of 0.7-5% (w/w), such as in the range of 0.8-3% (w/w), e.g. in the range of 0.9-1.5% (w/w), such as about 1% (w/w).

The feed product is preferably constructed with a sufficient amount of oxygen agent to provide in the range of 10-1000 L oxygen (preferably dioxygen (O₂)) per day in the gastrointestinal tract, e.g. the rumen, of a ruminant, such as in the range of 25-750 L oxygen (preferably dioxygen (O₂)) per day, e.g. in the range of 50-500 L oxygen (preferably dioxygen (O₂)) per day, such as in the range of 75-400 L oxygen (preferably dioxygen (O₂)) per day, e.g. in the range of 80-300 L oxygen (preferably dioxygen (O₂)) per day, such as in the range of 90-200 L oxygen (preferably dioxygen (O₂)) per day, e.g. in the range of 100-150 L oxygen (preferably dioxygen (O₂)) per day, such as in the range of 150-400 L oxygen (preferably dioxygen (O₂)) per day, e.g. in the range of 200-300 L oxygen (preferably dioxygen (O₂)) per day.

A preferred embodiment of the present invention relates to a feed product comprising a viable methanotroph in combination with at least one oxygen agent, wherein the oxygen agent is provided in an amount sufficient to reduce at least 10% of the methane generated in the gastrointestinal tract, in particular the rumen, of a ruminant, such as at least 20%, e.g. at least 30%, such as at least 40%, e.g. at least 50%, such as at least 60%, e.g. at least 70%, such as at least 80%, e.g. at least 90%, such as at least 95%, e.g. at least 98%.

The feed product may comprise 1-15% (w/w) methanotroph (preferably, viable methanotroph), e.g. 2-12% (w/w) methanotroph (preferably, viable methanotroph); such as 3-10% (w/w) methanotroph (preferably, viable methanotroph); e.g. 4-8% (w/w) methanotroph (preferably, viable methanotroph), such as about 5% (w/w) methanotroph (preferably, viable methanotroph); and in the range of 0.1-25% (w/w) oxygen agent, in particular an oxygen-precursor compound, preferably, a hydrogen peroxide (H₂O₂) or a hydrogen peroxide generating compound (preferably a percarbonate like sodium percarbonate) relative to the weight of the feed product, such as in the range of 0.2-20% (w/w), e.g. in the range of 0.3-15% (w/w), such as in the range of 0.4-10% (w/w), e.g. in the range of 0.5-8% (w/w), such as in the range of 0.6-6% (w/w), e.g. in the range of 0.7-5% (w/w), such as in the range of 0.8-3% (w/w), e.g. in the range of 0.9-1.5% (w/w), such as about 1% (w/w).

In the context of the present invention, the term “feed” relates to a foodstuff provided for animal consumption and may be either a complete feed or one or more parts of a complete feed. The feed product may comprise a basic feed material in combination with the ingredient according to the present invention.

In an embodiment of the present invention basic feed material may comprise a dry basic feed, a semi dry basic feed, or a wet basic feed.

The basic feed material according to the present invention may comprise a mineral mixture, a vitamin premix, a cereal mixture, a plant seed mixture or combinations hereof.

In an embodiment of the present invention the feed product further comprises a cereal. The cereal may be selected from rye, maize, wheat, barley, oat, or a combination hereof.

The feed product according to the present invention may further comprise a plant material.

Preferably, the plant material may be selected from soybean, rape, grass, maize, like maize stalks, potato, potato plants, potato tops, alfalfa, clovers, peas and/or beans.

The feed product according to the present invention may further comprises at least one protein source. In an embodiment of the present invention the at least one protein source comprises a single cell protein (SCP) or a fraction thereof.

The feed product according to the present invention may comprise at least 0.1% (w/w) single cell protein (SCP), such as at least 0.25% (w/w), e.g. at least 0.5% (w/w), such as at least 0.75% (w/w), e.g. at least 1.0% (w/w), such as at least 1.5% (w/w), e.g. at least 2.0% (w/w), such as at least 2.5% (w/w), e.g. at least 3.0% (w/w), such as at least 4.0% (w/w), e.g. at least 5.0% (w/w).

Preferably, the feed product according to the present invention the feed product may comprise in the range of 0.1-6% (w/w) single cell protein (SCP), such as in the range of 0.25-5.5% (w/w), e.g. in the range of 0.5-5.0% (w/w), such as in the range of 0.75-4.5% (w/w), e.g. in the range of 1.0-4% (w/w), such as in the range of 1.5-3% (w/w), e.g. at least 1.8-2.5% (w/w), such as in the range of 1.9-2.2% (w/w), e.g. about 2.0% (w/w).

A feed for ruminants, such as cattle, may comprises organic materials including at least one protein source, at least one carbohydrate source, at least one lipid source, at least one mineral source, at least one fibrous source, at least one vitamin source.

The at least one carbohydrate source may include molasses or derivates thereof.

In an embodiment of the present invention the feed product comprise an ingredient according to the present invention and rye and/or maize in combination with a plant material, preferably soybean and/or rape, in combination with at least one lipid source, in particular palm fatty acid distillate and/or palm-oil cakes.

The ruminant may be selected from the group consisting of a cattle, a cow, a goat, a sheep, a buffalo, a giraffe, a deer, a gazelle, and an antelope.

The inventor of the present invention surprisingly found that by supplying an oxygen agent to the animal, in particular to the rumen of a ruminant, not only a reducing effect on emission of methane from the ruminant for the environmental benefit may be provided, but also a significant reduction in the emission of carbon dioxide (CO₂) may be observed.

Without being bound by theory, the inventor of the present invention trusts that the oxygen agent in combination with the carbon dioxide (CO₂), methane (CH₄) and a nitrogen compound in the rumen or optionally in the feed, may the react forming a peptide or a protein. In an embodiment of the present invention, the peptide or the protein formed may act as a protein source digested by the animal, in particular the ruminant.

The present invention relates to the use of the ingredient according to the present invention and/or use of the feed product according to the present invention for increasing the milk production in a ruminant.

Ruminants feed with the ingredient and/or the feed product according to the present invention may increase in milk production by at least 2%, such as at least 5%, e.g. at least 7.5% e.g. at least 10%, such as at least 15%, e.g. at least 20%, relative to the yield of milk obtained when using traditional feed products (a feed product without addition of the ingredient according to the present invention).

A preferred embodiment of the present invention relates to the use of the ingredient according to the present invention and/or use of the feed product according to the present invention for increasing the milk production in a ruminant.

A further preferred embodiment of the present invention relates to a method for increasing the milk production in a ruminant by administering the ingredient according to the present invention and/or the feed product according to the present invention to an animal, preferably to a ruminant.

It should be noted that embodiments and features described in the context of one of the aspects of the present invention also apply to the other aspects of the invention.

1. An ingredient, wherein the ingredient comprises a viable methanotroph in combination with at least one oxygen agent.

2. The ingredient according to claim 1, wherein the oxygen agent is a readily available oxygen compound, an oxygen-precursor compound, an oxygen-developing agent, or a combination hereof.

3. The ingredient according to claim 1, wherein the viable methanotroph constitutes at least 40% of the total number of viable microorganisms in the ingredient (based on cell count), such as at least 50%, e.g. at least 60%, such as at least 70%, e.g. at least 80%, such as at least 90%, e.g. at least 95%, such as at least 98%, e.g. at least 99%.

4. The ingredient according to claim 1, wherein the ingredient further comprises a single cell protein (SCP) or a derivative hereof.

5. The ingredient according to claim 1, wherein the ingredient comprises a pH in the range of pH 5.5-9.0, such as in the range of pH 6.0-8.5, e.g. in the range of pH 6.5-8.0, such as in the range of pH 6.8-7.5.

6. The ingredient according to claim 1, wherein the ingredient may be a liquid ingredient, an aqueous ingredient or a dry ingredient.

7. A feed product comprising a viable methanotroph.

8. A feed product comprising the ingredient according to claim 1.

9. The feed product according to claim 7, wherein the feed product comprises 1-15% (w/w) of the ingredient; such as 3-10% (w/w); e.g. 4-8% (w/w), such as about 5% (w/w).

10. The feed product according to claim 7, wherein the feed product comprises 1-15% (w/w) methanotroph, e.g. 2-12% (w/w) methanotroph; such as 3-10% (w/w) methanotroph; e.g. 4-8% (w/w) methanotroph, such as about 5% (w/w) methanotroph.

11. A feed product comprising an oxygen agent.

12. The feed product according to claim 8, wherein the oxygen agent is an encapsulated oxygen agent.

13. The feed product according to claim 12, wherein the encapsulated oxygen agent may provide a controlled release of the oxygen agent (to the gastrointestinal tract, in particular the rumen, of the ruminant) over a period of 1-12 hours, such as over a period of 1.5-8 hours, e.g. over a period of 2-6 hours, such as over a period of 2.5-4 hours, e.g. over a period of 2.75-3 hours.

14. The feed product according to claim 8, wherein the feed product is provided with 0.0001-0.3 kg oxygen agent, in particular an oxygen-precursor compound, preferably, a hydrogen peroxide (H₂O₂) or a hydrogen peroxide generating compound (preferably a percarbonate like sodium percarbonate) per kg feed.

15. The feed product according to claim 8, wherein the feed product is constructed with a sufficient amount of oxygen agent to provide in the range of 10-1000 L oxygen (preferably dioxygen (O₂)) per day in the gastrointestinal tract, e.g. the rumen, of a ruminant.
